(19) United States
(12) Patent Application Publication
(10) Pub. No.: US 2025/0241623 A1
(43) Pub. Date: Jul. 31, 2025

(54) ULTRASONIC IMAGING APPARATUS

(71) Applicant: SHENZHEN MINDRAY BIO-MEDICAL ELECTRONICS CO., LTD., Shenzhen (CN)

(72) Inventors: Zhian XU, Shenzhen (CN); Tengju DONG, Shenzhen (CN); Yuan XUE, Shenzhen (CN)

(73) Assignee: SHENZHEN MINDRAY BIO-MEDICAL ELECTRONICS CO., LTD., Shenzhen (CN)

(21) Appl. No.: 19/034,527

(22) Filed: Jan. 22, 2025

(30) Foreign Application Priority Data
Jan. 26, 2024 (CN) 202410116434.1

(51) Int. Cl. A61B 8/00

(52) U.S. Cl. CPC A61B 8/5207; A61B 8/4483; A61B 8/54

(57) ABSTRACT

Disclosed is an ultrasonic imaging apparatus featuring two imaging modes, under the first imaging mode: the transmission control circuit controls transducer elements with first center frequency to operate as transmitting elements to emit ultrasonic waves to a first region; and under the second imaging mode: the transmission control circuit controls both the transducer elements with first center frequency and the transducer elements with second center frequency to jointly operate as transmitting elements to transmit ultrasonic waves to a second region.

### TECHNICAL FIELD

The present disclosure relates to ultrasonic imaging, in particular to ultrasonic imaging apparatus.

### BACKGROUND

Ultrasonic imaging technology enables the imaging of numerous organs within the human body, aiding doctors in diagnosis. This technology involves scanning human tissues and organs using ultrasonic waves, and obtaining images of the corresponding regions through the reception and processing of reflected signals.

The ultrasonic probe is an essential and crucial component in an ultrasonic imaging device. It serves to convert electrical signals into ultrasonic signals and vice versa, possessing dual functions of ultrasonic transmission and reception. Generally, different ultrasonic probes are required for scanning various human tissues and parts to adapt to the corresponding tissues and areas being scanned, which brings significant inconvenience to daily use.

### SUMMARY

The present disclosure provides ultrasonic imaging apparatus, which will be specifically described below.

In one embodiment, an ultrasonic imaging apparatus is provided, which may include:
an ultrasonic probe comprising a transducer array having a plurality of transducer elements with first center frequency and a plurality of transducer elements with second center frequency;
a transmission control circuit configured to control the transducer array to transmit ultrasonic waves;
a reception control circuit configured to control the transducer array to receive echo signals from ultrasonic waves; and
a processor configured to generate an ultrasonic image based on the echo signals;
where the ultrasonic imaging apparatus may operate in a first imaging mode and a second imaging mode;
under the first imaging mode: the transmission control circuit controls the transducer elements with first center frequency to operate as transmitting elements to transmit ultrasonic waves to a first region, the reception control circuit controls part or all of transducer elements within the transducer array to operate as receiving elements to receive echo signals from the first region; and the processor generates an ultrasonic image for the first region based on the received echo signals; and
under the second imaging mode: the transmission control circuit controls both the transducer elements with first center frequency and the transducer elements with second center frequency to jointly operate as transmitting elements to transmit ultrasonic waves to a second region, the reception control circuit controls part or all of transducer elements within the transducer array to operate as receiving elements to receive echo signals from the second region, and the processor generates an ultrasonic image for the second region based on the received echo signals.

In some embodiments, the second region comprises a first sub-region proximal to the transducer array and a second sub-region distal to the transducer array; and
under the second imaging mode: the transmission control circuit controls the transducer elements with first center frequency to operate as the transmitting elements to transmit ultrasonic waves to the first sub-region, and controls the transducer elements with second center frequency to operate as the transmitting elements to transmit ultrasonic waves to the second sub-region.

In some embodiments, under the second imaging mode: the reception control circuit controls the transducer elements with first center frequency to operate as the receiving elements to receive echo signals from the first sub-region, and controls the transducer elements with second center frequency to operate as the receiving elements to receive echo signals from the second sub-region.

In some embodiments, the imaging depth of the first region is shallower than that of the second region, and the imaging depth refers to either an average depth or a maximum depth.

In some embodiments, under the first imaging mode: the reception control circuit controls the transducer elements with first center frequency to operate as the receiving elements to receive the echo signals from the first region; and/or under the second imaging mode: the reception control circuit controls the transducer elements with first center frequency and the transducer elements with second center frequency to jointly operate as the receiving elements to receive the echo signals from the second region.

In some embodiments, under the first imaging mode, the transmission control circuit generates excitation signals in a first frequency range to activate the transducer elements with first center frequency; and/or,
under the second imaging mode, the transmission control circuit generates excitation signals in a second frequency range to activate the transducer elements with first center frequency and the transducer elements with second center frequency; or under the second imaging mode, the transmission control circuit generates excitation signals in a first frequency range to activate the transducer elements with first center frequency, and excitation signals in a second frequency range to activate transducer elements with second center frequency.

In some embodiments, the ultrasonic imaging apparatus further operates in a third imaging mode; the transmission control circuit controls the transducer elements with second center frequency to operate as the transmitting elements to transmit ultrasonic waves to a third region, the reception control circuit controls part or all of transducer elements within the transducer array to operate as the receiving elements to receive echo signals from the third region, and the processor generates an ultrasonic image for the third region based on the echo signals from the third region.

In some embodiments, the imaging depth of the first region is shallower than that of the third region, and the imaging depth refer to an average depth, a maximum depth or a minimum depth.

In some embodiments, under the third imaging mode, the transmission control circuit generates excitation signals in a second frequency range to activate the transducer elements with second center frequency.

In some embodiments, the processor is capable of automatically setting a current imaging mode.

In some embodiments, the processor obtains a current scanning mode and automatically sets the current imaging mode based on the current scanning mode; and/or, the processor obtains current imaging parameters and automatically sets the current imaging mode based on the current imaging parameters, the imaging parameters at least comprising an imaging depth; and/or, the processor automatically identifies scanned region based on a current ultrasonic image and automatically switches the current imaging mode based on the identified scanned region.

In some embodiments, the transducer array comprises an array of transducer elements arranged in M rows and N columns, where M is an integer ≥3, N is an integer ≥2; where M is an odd number, and for each column of transducer elements: the transducer element positioned in the (M+1)/2-th row is individually wired to a channel to receive excitation signals, while the transducer elements that are symmetrically positioned around the element positioned in the (M+2)/2-th row of that column are paired, connected together, and then wired to receive excitation signals from the same channel; and for this M×N array, the transducer elements in the (M+1)/2-th row are the transducer elements with first center frequency, and the transducer elements in the 1^{st} and M-th rows are the transducer elements with second center frequency.

In some embodiments, the transducer elements from the 1^{st} to the m-th rows and from the (M+1−m)-th to the M-th rows are the transducer elements with second center frequency, and the transducer elements from the (m+1)-th to the (M−m)-th rows are the transducer elements with first center frequency, where m is a positive integer and less than (M+1)/2.

In some embodiments, the transducer array comprises an array of elements arranged in M rows and N columns, where M is an integer ≥3, N is an integer ≥2; where M is an even number, and for each column of transducer elements: the transducer elements symmetrically positioned in that column are paired, connected together, and then wired to receive excitation signals from a same channel; and for this M×N array, the transducer elements in the M/2-th row and the (M/2+1)-th row are the transducer elements with first center frequency, and the transducer elements in the 1st and M-th rows are the transducer elements with second center frequency.

In some embodiments, the transducer elements from the 1^{st} to the m-th rows and from the (M+1−m)-th to the M-th rows are the transducer elements with second center frequency, and the transducer elements from the (m+1)-th to the (M−m)-th rows are the transducer elements with first center frequency, where m is a positive integer and less than (M+1)/2.

In some embodiments, the ultrasonic probe is a 1.XD probe, where X is a specific numerical value greater than 0 and less than 2; more preferably, the 1.XD probe is selected from a group consisting of a 1.25D probe, a 1.5D probe and a 1.75D probe.

In some embodiments, the transducer elements with first center frequency and the transducer elements with second center frequency are spaced apart and aligned in a row.

In some embodiments, the first center frequency is higher than the second center frequency.

In some embodiments, the first center frequency is greater than 7 MHz and less than 15 MHz; and/or the second center frequency is greater than 2 MHz and less than 4 MHz.

In one embodiment, an ultrasonic imaging apparatus is provided, which may include:
an ultrasonic probe comprising a transducer array having an array of elements arranged in M rows and N columns, where for each column of transducers elements: the transducer elements symmetrically positioned in that column are paired, connected together, and then wired to a channel to receive excitation signals, and when there is a single central transducer element in that column, the central transducer element is individually wired to a channel to receive excitation signals; and for this M×N array, there are a plurality of transducer elements with first center frequency and a plurality of transducer elements with second center frequency, and the transducer elements connected to the same wire are of the same center frequency;
a transmission control circuit configured to control the transducer array to transmit ultrasonic waves;
a reception control circuit configured to control the transducer array to receive echo signals from the ultrasonic waves; and
a processor configured to generate an ultrasonic image based on the echo signals.

In some embodiments, for this M×N array, some rows of the transducer elements are the transducer elements with first center frequency, and other rows of the transducer elements are the transducer elements with second center frequency.

In some embodiments, M is an odd number, for each column of transducer elements: the transducer element positioned in the (M+1)/2-th row is a central transducer element of that column and is individually wired to a channel to receive excitation signals, and the transducer elements in that column that are symmetrically positioned around the central transducer element are paired, connected together, and then wired to receive excitation signals from the same channel; and for this M×N array, the transducer elements in the (M+1)/2-th row are the transducer elements with first center frequency, and the transducer elements in the 1st and M-th rows are the transducer elements with second center frequency.

In some embodiments, the transducer elements from the 1st to the m-th rows and from the (M+1−m)-th to the M-th rows are the transducer elements with second center frequency, and the transducer elements from the (m+1)-th to the (M−m)-th rows are the transducer elements with first center frequency, where m is a positive integer and less than (M+1)/2.

In some embodiments, M is an even number, for each column of transducer elements: the transducer elements symmetrically positioned in that column are paired, connected together, and then wired to receive excitation signals from a same channel; and for this M×N array, the transducer elements in the M/2-th row and the (M/2+1)-th row are the transducer elements with first center frequency, and the transducer elements in the 1st and M-th rows are the transducer elements with second center frequency.

In some embodiments, the transducer elements from the 1st to the m-th rows and from the (M+1−m)-th to the M-th rows are the transducer elements with second center frequency, and the transducer elements from the (m+1)-th to the (M−m)-th rows are the transducer elements with first center frequency, where m is a positive integer and less than (M+1)/2.

In some embodiments, the ultrasonic probe is a 1.XD probe, where X is a specific numerical value greater than 0 and less than 2; more preferably, the 1.XD probe is selected from a group consisting of a 1.25D probe, a 1.5D probe and a 1.75D probe.

In some embodiments, the first center frequency is higher than the second center frequency.

In some embodiments, the first center frequency is greater than 7 MHz and less than 15 MHz; and/or the second center frequency is greater than 2 MHz and less than 4 MHz.

In some embodiments, the ultrasonic imaging apparatus operates in a first imaging mode and a second imaging mode;
under the first imaging mode: the transmission control circuit controls the transducer elements with first center frequency to operate as transmitting elements to transmit ultrasonic waves to a first region, the reception control circuit controls part or all of transducer elements within the transducer array to operate as receiving elements to receive echo signals from the first region; and the processor generates an ultrasonic image for the first region based on the received echo signals; and
under the second imaging mode: the transmission control circuit controls both the transducer elements with first center frequency and the transducer elements with second center frequency to jointly operate as transmitting elements to transmit ultrasonic waves to a second region, the reception control circuit controls part or all of transducer elements within the transducer array to operate as receiving elements to receive echo signals from the second region, and the processor generates an ultrasonic image for the second region based on the received echo signals.

In some embodiments, the second region comprises a first sub-region proximal to the transducer array and a second sub-region distal to the transducer array; and
under the second imaging mode: the transmission control circuit controls the transducer elements with first center frequency to operate as the transmitting elements to transmit ultrasonic waves to the first sub-region, and controls the transducer elements with second center frequency to operate as the transmitting elements to transmit ultrasonic waves to the second sub-region.

In some embodiments, under the second imaging mode: the reception control circuit controls the transducer elements with first center frequency to operate as the receiving elements to receive echo signals from the first sub-region, and controls the transducer elements with second center frequency to operate as the receiving elements to receive echo signals from the second sub-region.

In some embodiments, under the first imaging mode: the reception control circuit controls the transducer elements with first center frequency to operate as the receiving elements to receive the echo signals from the first region; and/or under the second imaging mode: the reception control circuit controls the transducer elements with first center frequency and the transducer elements with second center frequency to jointly operate as the receiving elements to receive the echo signals from the second region.

In some embodiments, the processor obtains a current scanning mode and automatically sets the current imaging mode based on the current scanning mode; and/or, the processor obtains current imaging parameters and automatically sets the current imaging mode based on the current imaging parameters, the imaging parameters at least comprising an imaging depth; and/or, the processor automatically identifies scanned region based on a current ultrasonic image and automatically switches the current imaging mode based on the identified scanned region.

According to the ultrasonic imaging apparatus in the aforesaid embodiments, there are two imaging modes, where under the first imaging mode: the transmission control circuit can control the transducer elements with first center frequency to operate as transmitting elements to transmit ultrasonic waves to the first region; and under the second imaging mode: the transmission control circuit can control the transducer elements with first center frequency and the transducer elements with second center frequency to jointly operate as transmitting elements to transmit ultrasonic waves to a second region. By incorporating transducer elements of different frequencies into a single ultrasonic probe, two distinct imaging modes are introduced, where the first imaging mode offers higher image resolution, while the second imaging mode provides stronger spatial penetration of ultrasonic waves, making them suitable for various scenarios and requirements.

According to the ultrasonic imaging apparatus in the aforesaid embodiments, the ultrasonic probe comprises a transducer array having an array of elements arranged in M rows and N columns, where M is an integer ≥3, and N is an integer ≥2; for each column of transducer elements, elements symmetrically positioned in that column are paired, connected together, and then wired to receive excitation signals from the same channel, and when there is a single central transducer element in that column, it is separately wired to connect to a channel to receive excitation signals; and for this M×N array, there are a plurality of transducer elements with first center frequency and a plurality of transducer elements with second center frequency, and the transducer elements connected to the same wire are of the same center frequency. This configuration of the ultrasonic probe enables it to balance the resolution of near-field images with the penetration required for far-field images.

### DESCRIPTION OF THE DRAWINGS

FIG. 1 is a schematic structural diagram of an ultrasonic imaging apparatus according to some embodiment;

FIG. 2 is a schematic structural diagram of an ultrasonic probe according to some embodiment;

FIG. 3 is a schematic structural diagram of a transducer array according to some embodiment;

FIG. 4 is a schematic structural diagram of a transducer array according to some embodiment;

FIG. 5 is a schematic structural diagram of a transducer array according to some embodiment;

FIG. 6 is a schematic structural diagram of a transducer array according to some embodiment;

FIG. 7 is a schematic structural diagram of a transducer array according to some embodiment;

FIG. 8 is a schematic structural diagram of a transducer array according to some embodiment;

FIG. 9 is a schematic structural diagram of a transducer array according to some embodiment;

FIG. 10 is a schematic structural diagram of a transducer array according to some embodiment;

FIG. 11 is a schematic diagram of a first imaging mode according to some embodiment;

FIG. 12 is a schematic diagram of a second imaging mode according to some embodiment; and

FIG. 13 is a schematic diagram of a third imaging mode according to some embodiment.

### DETAILED DESCRIPTION

The present disclosure will be further described in detail below through specific embodiments with reference to the accompanying drawings. Common or similar elements are referenced with like or identical reference numerals in different embodiments. Many details described in the following embodiments are for better understanding the present disclosure. However, those skilled in the art can realize with minimal effort that some of these features can be omitted in different cases or be replaced by other elements, materials and methods. For clarity some operations related to the present disclosure are not shown or illustrated herein so as to prevent the core from being overwhelmed by excessive descriptions. For those skilled in the art, such operations are not necessary to be explained in detail, and they can fully understand the related operations according to the description in the specification and the general technical knowledge in the art.

In addition, the features, operations or characteristics described in the specification may be combined in any suitable manner to form various embodiments. At the same time, the steps or actions in the described method can also be sequentially changed or adjusted in a manner that can be apparent to those skilled in the art. Therefore, the various sequences in the specification and the drawings are only for the purpose of describing a particular embodiment, and are not intended to be an order of necessity, unless otherwise stated one of the sequences must be followed.

The serial numbers of components herein, such as “first”, “second coupled” etc., are only used to distinguish the described objects and do not have any order or technical meaning. The terms “connected”, “coupled” and the like here include direct and indirect connections (coupling) unless otherwise specified.

An ultrasonic scanning procedure may be conducted as follows: a patient is positioned either in a seated or lying posture; and a physician holds an ultrasonic probe and positions it in close proximity to the patient's body surface, emitting ultrasonic waves to the region of interest beneath the surface. These waves penetrate through human tissues and reach the target region. The ultrasonic waves reflected from the region of interest are captured by the ultrasonic probe and subsequently processed by the system to generate an ultrasonic image.

The penetration capability of ultrasonic waves is correlated with their frequency. Specifically, lower frequencies and longer wavelengths yield greater penetration depth, albeit at the expense of reduced spatial resolution in the resulting ultrasonic image. Conversely, higher frequencies and shorter wavelengths result in weaker penetration but offer enhanced spatial resolution. Currently, linear array probes are typically employed for imaging the near field close to the probe, while convex array probes are used for imaging the far field further away from the probe within the human body.

An ongoing improvement focus for ultrasonic probes is to develop a single probe capable of adapting to a wide range of imaging scenarios. Ideally, such a probe would facilitate both near-field and far-field imaging through a single device.

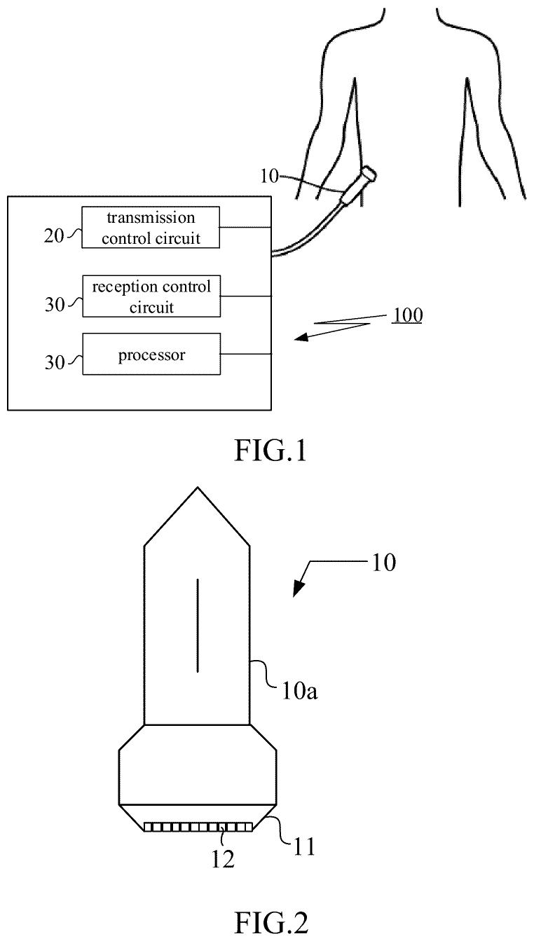

Referring to FIG. 1, an ultrasonic imaging apparatus 100 in some embodiments comprises an ultrasonic probe 10, a transmission control circuit 20, a reception control circuit 30 and a processor 40, which are described below.

In some embodiments, the ultrasonic probe 10 may be configured to transmit and receive ultrasonic waves. In some embodiments, the ultrasonic probe 10 may include a transducer array 11. In some embodiments, the transducer array 11 may include a plurality of transducer elements 12. FIG. 2 illustrates an example, wherein the ultrasonic probe 10 includes a probe body 10a and a transducer array 11 arranged on the probe body 10a.

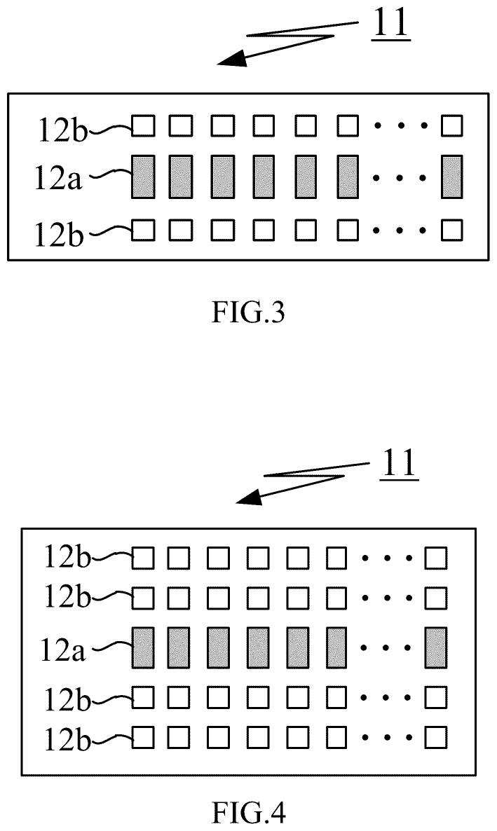

In some embodiments, the transducer array 11 may comprise transducer elements 12 of at least two different frequencies. For example, the transducer array 11 includes a plurality of transducer elements with first center frequency 12a and a plurality of transducer elements with second center frequency 12b (FIGS. 3, 4, 5, and 6 below provide relevant examples). It should be understood that “a plurality of” herein means an indefinite number, i.e., one or more. In some embodiments, the transducer elements 12 may be configured to facilitate the reciprocal conversion between electrical signals and ultrasonic waves, enabling the emission of ultrasonic waves towards a target region and the reception of echo signals reflected back by the target tissues. The transducer elements may be implemented using piezoelectric ceramics, piezoelectric crystals, or piezoelectric composite materials, for instance. The transducer elements may be configured to emit ultrasonic waves in response to excitation electrical signals or to convert received ultrasonic waves into electrical signals. Consequently, each transducer element may be capable of facilitating the reciprocal conversion between electrical pulse signals and ultrasonic waves, thereby enabling the emission of ultrasonic waves towards a target region and the reception of ultrasonic echo signals reflected back by tissues.

In some embodiments, the transducer array 11 may comprise an array of elements arranged in M rows and N columns, where M is an integer ≥3, and N is an integer ≥2. In some embodiments, for the transducer elements 12 in each column: the transducer elements 12 symmetrically positioned in that column are paired, connected together, and then wired to receive excitation signals from the same channel 01; and when there exists a single central transducer element 12 in that column, this central transducer element 12 is individually wired to connect to a channel to receive excitation signals from the channel 01. For the array of elements arranged in M rows and N columns 12, there are a plurality of transducer elements with first center frequency 12a and a plurality of transducer elements with second center frequency 12b, and the transducer elements 12 connected to the same wire are of the same center frequency.

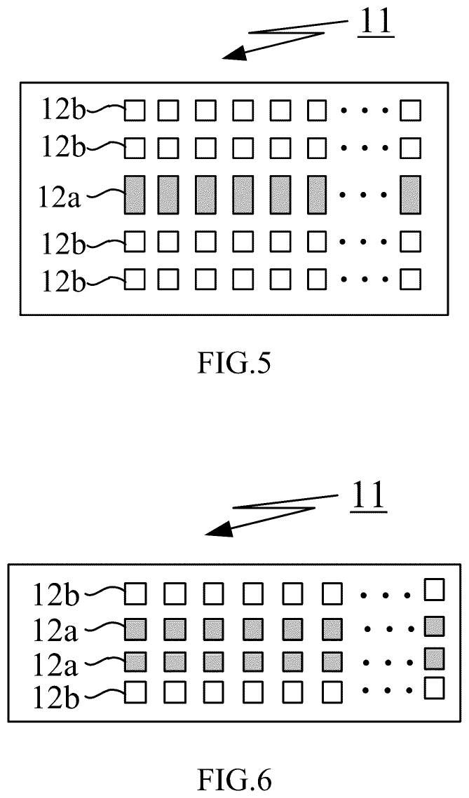

In some embodiments, for the array of elements arranged in M rows and N columns 12, some rows of transducer elements 12 are the transducer elements with first center frequency 12a, and some other rows of transducer elements 12 are the transducer elements with second center frequency 12b. That is, for the array of elements arranged in M rows and N columns 12, there is one or more rows of transducer elements with first center frequency 12a and one or more rows of transducer elements with second center frequency 12b. FIGS. 3-6 illustrate four examples, where the gray-filled squares represent the transducer elements with first center frequency 12a and white-filled squares represent the transducer elements with second center frequency 12b. In the example shown in FIG. 3, M=3, meaning the transducer array 11 has three rows of elements; and the second row of elements located centrally are the transducer elements with first center frequency 12a, while the symmetrical first and third rows of elements are the transducer elements with second center frequency 12b. In the example shown in FIG. 4, M=5, meaning the transducer array 11 has five rows of elements; and the third row of elements located centrally are the transducer elements with first center frequency 12a, while the symmetrical first and fifth rows of elements, as well as the symmetrical second and fourth rows of elements, are the transducer elements with second center frequency 12b. In the example shown in FIG. 5, M=5, meaning the transducer array 11 has five rows of elements; and the third row of elements located centrally, as well as the symmetrical second and fourth rows of elements, are the transducer elements with first center frequency 12a, while the symmetrical first and fifth rows of elements are the transducer elements with second center frequency 12b. In the example shown in FIG. 6, M=4, meaning the transducer array 11 has four rows of elements; and the symmetrical second and third rows of elements are the transducer elements with first center frequency 12a, while the symmetrical first and fourth rows of elements are the transducer elements with second center frequency 12b. The term “symmetrical” here refers to symmetry in the column direction.

In some embodiments, M is an odd number; for the array of elements arranged in M rows and N columns 12, the transducer elements 12 in the (M+1)/2-th row are the transducer elements with first center frequency 12a, while the transducer elements 12 in the 1st and M-th rows are the transducer elements with second center frequency 12b. In some embodiments, the transducer elements 12 from the 1st to m-th rows, as well as the elements 12 from the (M+1−m)-th to the m-th rows, are the transducer elements with second center frequency 12b, while the elements 12 from the (m+1)-th to (M−m)-th rows are the transducer elements with first center frequency 12a, where m is a positive integer and less than (M+1)/2.

In some embodiments, M is an odd number. When M is odd, the transducer elements 12 in the (M+1)/2-th row, which is the central row of the array, are designated as the central transducer elements. For each column of transducer elements 12, the elements in the (M+1)/2-th row of that column is the central transducer elements of that column. Accordingly, for each column of the transducer elements 12: the elements 12 in the (M+1)/2-th row within that column is the central transducer elements, and it is individually connected to a wire to connect to a channel 01 and receive excitation signals from that channel 01; the elements 12 in that column that are symmetrically positioned around the central transducer elements, i.e., the (M+1)/2-th row, are paired and connected to a wire to receive excitation signals from the same channel 01. That is, the rows numbered from 1 to [(M+1)/2−1] in ascending order are symmetrically paired with the rows numbered from M to [(M+1)/2+1] in descending order.

In some embodiments, the transducer elements 12 arranged in symmetric rows are of the same center frequency.

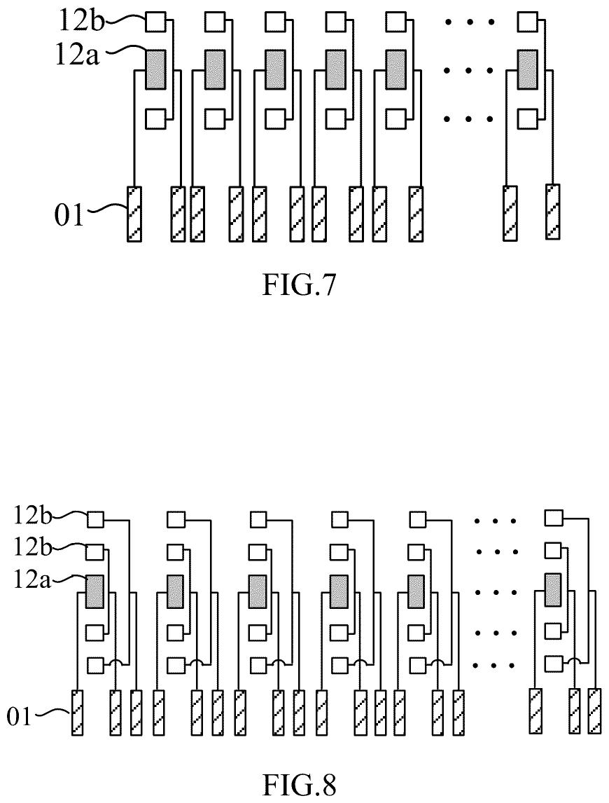

FIGS. 7-8 illustrate two examples, where gray-filled squares represent the transducer elements with first center frequency 12a, white-filled squares represent the transducer elements with second center frequency 12b, and diagonally filled rectangles represent the channels 01.

In the example shown in FIG. 7, M=3, meaning the transducer array 11 comprises three rows of transducer elements, with the transducer elements 12 in the second row being the central transducer elements. For each column of transducer elements 12, the second-row elements in that column is the central transducer elements. Consequently, the second-row elements in every column are individually wired to connect to a channel 01 to receive excitation signals from that channel 01. For instance, for the three (rows of) transducer elements in the first column, the element 12 in the second row is the central transducer element, which is individually wired to connect to a channel 01 to receive excitation signals from that channel 01. Similarly, in the three (rows of) transducer elements in the second column, the element 12 in the second row is the central transducer element, which is individually wired to connect to a channel 01 to receive excitation signals from that channel 01. Still similarly, in the three (rows of) transducer elements in the n-th column, the element 12 in the second row is the central transducer element, which is individually wired to connect to a channel 01 to receive excitation signals from that channel 01, where n is a positive integer less than or equal to N. For each column of transducer elements 12, the elements 12 that are symmetrically positioned around the second-row element in that column are paired, connected together, and then wired to receive excitation signals from the same channel 01. That is, the first-row and third-row elements 12 in each column are pairwise connected and then wired to receive excitation signals from the same channel 01. For example, in the three (rows of) transducer elements in the first column, the first-row and third-row elements are symmetric and thus connected together to receive excitation signals from the same channel 01. This pattern also applies to the second column and the n-th column; that is, in the three (rows of) transducer elements in the second column, the first-row and third-row elements are symmetric and thus connected together to receive excitation signals from the same channel 01; and in the three (rows of) transducer elements in the n-th column, the first-row and third-row elements are symmetric and thus connected together to receive excitation signals from the same channel 01, where n is a positive integer less than or equal to N.

In the example shown in FIG. 8, M=5, meaning the transducer array 11 comprises five rows of transducer elements. The transducer elements 12 in the third row are the central transducer elements. For each column of transducer elements 12, the third-row elements in that column is the central transducer elements. Consequently, each element in the third row in every column is individually wired to connect to a channel 01 to receive excitation signals from that channel 01. For instance, in the first column of five (rows of) array elements, the element 12 in the third row is the central element, which is singularly connected to a wire leading to a channel 01 to receive excitation signals. Similarly, in the second column, the element 12 in the third row is the central element, also singularly connected to a wire for the same purpose. This pattern continues up to the n-th column, where the element 12 in the third row is likewise the central element and singularly connected. Here, n is a positive integer less than or equal to N. For each column of transducer elements 12, the elements 12 that are symmetrically positioned around the central element (i.e., the element in the third row) in that column are pairwise connected and wired to receive excitation signals from the same channel 01. Specifically, the elements in the first and fifth rows of each column are paired and connected to a wire, as are the elements in the second and fourth rows. For example, in the first column, the elements in the first and fifth rows are symmetric and thus paired and connected to a wire to receive signals from one channel 01, and similarly for the elements in the second and fourth rows. This arrangement applies to all columns, up to the n-th column, where the same pairing and connection logic is followed for the elements in the first and fifth rows, as well as the second and fourth rows. Again, n is a positive integer less than or equal to N.

In some embodiments, M is an even number; for the array of elements 12 arranged in M rows and N columns, the transducer elements 12 in the M/2-th row and the (M/2+1)-th row are the transducer elements with first center frequency 12a, while the transducer elements 12 in the the 1st and M-th rows are transducer elements with second center frequency 12b. In some embodiments, the elements 12 in the 1st to m-th rows, as well as the elements in the (M+1−m)-th to M-th rows, are the transducer elements with second center frequency 12b, and the elements 12 in the (m+1)-th to (M−m)-th rows are transducer elements with first center frequency 12a, where m is a positive integer and less than M/2.

In some embodiments, M is an even number; for each column of the transducer elements 12: the transducer elements 12 positioned symmetrically in that column are pairwise connected and then lead out a single wire to receive excitation signals from the same channel 01. When M is an even number, the first row is symmetrical to the last row, the second row is symmetrical to the second-to-last row, and so on. That is, the rows numbered from 1 to M/2 (from smallest to largest) are symmetrical to the rows numbered from M to (M/2+1) (from largest to smallest), respectively.

In some embodiments, the transducer elements 12 arranged in symmetric rows are of the same center frequency. FIG. 9 illustrates an example, where the gray-filled squares represent transducer elements with first center frequency 12a, the white-filled squares represent transducer elements with second center frequency 12b, and the diagonally-filled rectangles represent channels 01. In the example shown in FIG. 9, M=4, meaning the transducer array 11 consists of four rows of transducer elements, with the first and second rows of transducer elements being symmetrical, and the second and third rows of transducer elements being also symmetrical. For the transducer elements 12 in each column, the symmetrical transducer elements 12 are pairwise connected and then wired to receive excitation signals from the same channel 01. Specifically, for each column, the transducer elements 12 in the first and fourth rows are pairwise connected and then wired to receive excitation signals from the same channel 01, and the transducer elements 12 in the second and third rows are also pairwise connected and then wired to receive excitation signals from the same channel 01. For instance, in the four (rows of) transducer elements in the first column, the transducer elements in the first and fourth rows are symmetrical, so they are connected together and then wired to receive excitation signals from the same channel 01. Similarly, the transducer elements in the second and third rows in the first column are symmetrical, so they are connected together and then wired to receive excitation signals from the same channel 01. The same pattern applies to the four (rows of) transducer elements in the second column, and so on, up to the nth column, where n is a positive integer less than or equal to N. In the n-th column, the transducer elements in the first and fourth rows are symmetrical and connected together and then wired to receive signals from the same channel 01, and the transducer elements in the second and third rows are also symmetrical and connected together and then wired to receive signals from the same channel 01.

In some embodiments, the ultrasonic probe 10 is a 1.XD probe, where “X” represents a specific numerical value greater than 0 and less than 2, such as 1.25, 1.5, or 1.75. That is, the ultrasonic probe 10 may be a 1.25D probe, a 1.5D probe, or a 1.75D probe.

In some embodiments, the transducer array 11 of the ultrasonic probe 10 comprises a row of transducer elements 12, wherein the transducer elements with first center frequency 12a and the transducer elements with second center frequency 12b are arranged alternately; that is, the transducer elements with first center frequency 12a and the transducer elements with second center frequency 12b are spaced apart and aligned in a single row. FIG. 10 illustrates an example, where the gray-filled squares represent the transducer elements with first center frequency 12a, and the white-filled squares represent the transducer elements with second center frequency 12b. In such an example, the ultrasonic probe 10 may be a linear array probe.

The above provides some descriptions of the arrangement and connection relationships of the transducer array 11 in the ultrasonic probe 10. Furthermore, an explanation of the transducer elements with first center frequency 12a and the transducer elements with second center frequency 12b is provided.

In some embodiments, the first center frequency is higher than the second center frequency, and the first frequency range is higher than the second center frequency. Specifically, the first frequency range being higher than the second frequency range means that the minimum value in the first frequency range is greater than the maximum value in the second frequency range. Similarly, the first frequency range being higher than the second center frequency means that the minimum value in the first frequency range is higher than the second center frequency. Still similarly, the first center frequency being higher than the second frequency range means that the first center frequency is higher than the maximum value of that range. Similarly, the first center frequency being higher than the second frequency range means that the first center frequency is higher than the maximum value in the second frequency range.

In some embodiments, the first center frequency is greater than 7 MHz and less than 15 MHz.

In some embodiments, the first center frequency has a value ranging from 8 MHz to 10 MHz.

In some embodiments, the second center frequency is greater than 2 MHz and less than 4 MHz.

In some embodiments, the second center frequency has a value ranging from 3 MHz to 4 MHz.

The transducer elements with first center frequency 12a and the transducer elements with second center frequency 12b can be realized by using different materials and even different processes.

The above are some descriptions of the ultrasonic probe 10.

In some embodiments, the transmission control circuit 20 is configured to control the transducer array 11 to emit ultrasonic waves, while the reception control circuit 30 is configured to control the transducer array 11 to receive echo signals of ultrasonic waves.

In some embodiments, during ultrasonic inspection, the transmission control circuit 20 can control which elements are utilized to emit ultrasonic beams (transmitting elements), and the reception control circuit 30 can control which elements are utilized to receive ultrasonic beams (receiving elements), or control the elements to operate in time slots for emitting ultrasonic waves or receiving ultrasonic echoes. The transducer elements involved in ultrasonic emission (transmitting elements) can be simultaneously excited by electrical signals (which can be termed as excitation signals) to emit ultrasonic waves concurrently; alternatively, they can be excited by a plurality of electrical signals with specific time intervals to continuously emit ultrasonic waves with those intervals.

In some specific embodiments, the transmission control circuit 20 is configured to generate a transmit sequence and output it to the ultrasonic probe 10, while the reception control circuit 30 is configured to generate a receive sequence and output it to the ultrasonic probe 10. The transmit sequence is designed to control some or all of the elements in the transducer array 11 of the ultrasonic probe 10 to emit ultrasonic waves towards the region of interest. The parameters of the transmit sequence include the number of elements for transmission and ultrasonic emission parameters (such as amplitude, frequency, number of wave emissions, emission interval, emission angle, waveform, and/or focal position, etc.). The receive sequence is designed to control some or all of the elements in the transducer array 11 of the ultrasonic probe 10 to receive echoes of ultrasonic waves after they interact with tissue. The parameters of the receive sequence include the number of elements for reception and the reception parameters of the echoes (such as reception angle, depth, etc.). Depending on the intended use of the ultrasonic echo signals or the type of image generated from the ultrasonic echoes, the ultrasonic parameters in the transmit sequence and the echo parameters in the receive sequence may differ.

The processor 40 is configured to process the echo signals of ultrasonic waves received by the ultrasonic probe 10, performing one or more stages of data processing, such as receiving to form channel data, analog-to-digital conversion, signal demodulation, amplification, filtering, down-sampling, beamforming, modulus extraction, logarithmic compression, and grayscale transformation, etc. The processor 40 processes the ultrasonic echo signals received by the ultrasonic probe 10 to obtain an ultrasonic image of the target region. In some embodiments, the processor 40 includes, but is not limited to, devices for interpreting computer instructions and processing data in computer software, such as a Central Processing Unit (CPU), Micro Controller Unit (MCU), Field-Programmable Gate Array (FPGA), and Digital Signal Processing (DSP) unit.

In some embodiments, the processor 40 is also capable of executing various computer application programs stored in computer-readable storage media to carry out corresponding steps and methods.

In some embodiments, the processor 40 processes ultrasonic data by executing an ultrasonic imaging algorithm to generate an ultrasonic image.

The following is a description of an ultrasonic imaging apparatus 100. In some embodiments, the ultrasonic imaging apparatus 100 can operate in a first imaging mode and a second imaging mode, which are described below.

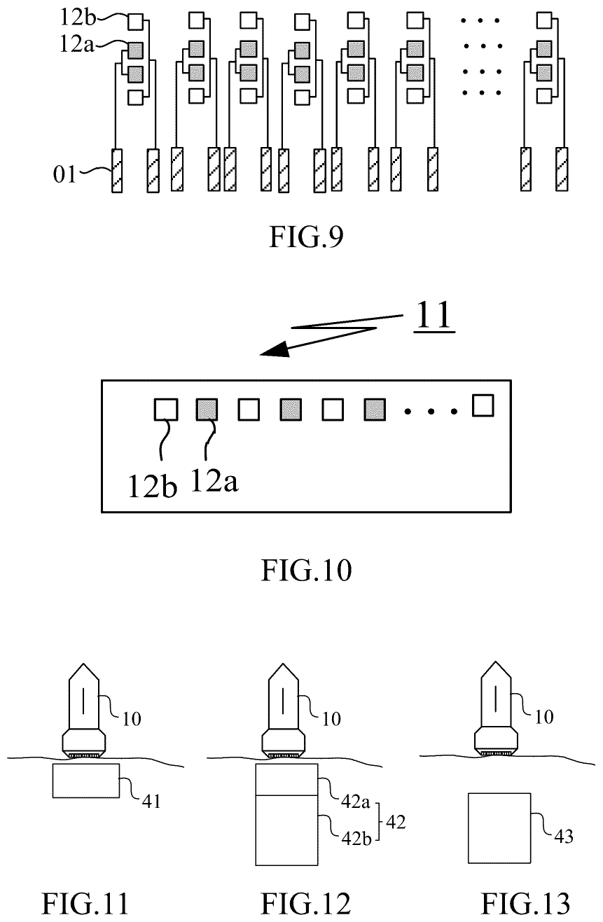

In some embodiment, under the first imaging mode:
the transmission control circuit 20 controls the transducer elements with first center frequency 12a to operate as transmitting elements for emitting ultrasonic waves to a first region 41 (FIG. 11 is an example); for instance, the transmission control circuit 20 generates excitation signals (electrical signals) in a first frequency range to excite the transducer elements with first center frequency 12a;
The reception control circuit 30 controls some or all of the transducer array 11 to operate as receiving elements for receiving echo signals from the first region 41; in some embodiments, the reception control circuit 30 controls the transducer elements with first center frequency 12a in the transducer array 11 to operate as receiving elements for receiving echo signals from the first region 41; and
the processor 40 generates an ultrasonic image for the first region based on the echo signals from the first region.

In some embodiment, under the second imaging mode:
the transmission control circuit 20 controls both the transducer elements with first center frequency 12a and the transducer elements with second center frequency 12b to operate as transmitting elements for emitting ultrasonic waves to a second region 42 (FIG. 12 is an example); for instance, the transmission control circuit 20 generates excitation signals (electrical signals) in a second frequency range to excite both the transducer elements with first center frequency 12a and the transducer elements with second center frequency 12b; alternatively, the transmission control circuit 20 generates excitation signals (electrical signals) in the first frequency range to excite the transducer elements with first center frequency 12a and excitation signals (electrical signals) within the second frequency range to excite the transducer elements with second center frequency 12b;
the reception control circuit 30 controls some or all of the transducer array 11 to operate as receiving elements for receiving echo signals from the second region 42; in some embodiments, the reception control circuit 30 controls both the transducer elements with first center frequency 12a and the transducer elements with second center frequency 12b in the transducer array 11 to operate as receiving elements for receiving echo signals from the second region 42; and
the processor 40 generates an ultrasonic image for the second region 42 based on the echo signals from the second region.

In some embodiments, the second region 42 includes a first sub-region 42a proximal to the transducer array 11 and a second sub-region 42b distal to the transducer array 11. The terms “proximal to” and “distal to” the transducer array 11 refer to the relative positions during the scanning process of the ultrasonic probe 10 (i.e., the process of emitting ultrasonic waves and receiving echo signals).

In some embodiments, the first sub-region 42a refers to the near-field region, and the second sub-region 42b refers to the far-field region.

In some embodiments, under the second imaging mode: the transmission control circuit 20 controls the transducer elements with first center frequency 12a to operate as transmitting elements for emitting ultrasonic waves to the first sub-region 42a and controls the transducer elements with second center frequency 12b to operate as transmitting elements for emitting ultrasonic waves to the second sub-region 42b; and in some embodiments, under the second imaging mode: the reception control circuit 30 controls the transducer elements with first center frequency 12a to operate as receiving elements for receiving echo signals from the first sub-region 42a and controls the transducer elements with second center frequency 12b to operate as receiving elements for receiving echo signals from the second sub-region 42b.

In some embodiments, the imaging depth of the first region 41 is less than that of the second region 42; and in some embodiments, the imaging depth refers to the average depth or maximum depth; for example, the average depth of the first region 41 is less than that of the second region 42, or the maximum depth of the first region 41 is less than that of the second region 42.

In some embodiments, the imaging depth of the first region 41 is the same as that of the first sub-region 42a; and in some embodiments, the imaging depth refers to the average depth or maximum depth.

In some embodiments, the imaging depth of the first region 41 is less than that of the second sub-region 42b; and in some embodiments, the imaging depth refers to the average depth, maximum depth, or minimum depth.

The above are some descriptions of the first and second imaging modes. The first imaging mode provides higher image resolution, while the second imaging mode offers stronger spatial penetration of ultrasonic waves, making them suitable for various scenarios and needs.

In some embodiments, the ultrasonic imaging apparatus 100 also has a third imaging mode. Under the third imaging mode:
the transmission control circuit 20 controls the second frequency element 12b to operate as transmitting elements for emitting ultrasonic waves to a third region 43 (FIG. 13 is an example); for instance, the transmission control circuit 20 generates excitation signals (electrical signals) in the second frequency range to excite the transducer elements with second center frequency 12b;
the reception control circuit 30 controls some or all of the transducer array 11 to operate as receiving elements for receiving echo signals from the third region 43; and in some embodiments, the reception control circuit 30 controls the transducer elements with second center frequency 12b in the transducer array 11 to operate as receiving elements for receiving echo signals from the third region 43; and
the processor 40 generates an ultrasonic image of the third region 43 based on the echo signals from the third region.

In some embodiments, the imaging depth of the first region 41 is less than that of the third region 43; and the imaging depth refers to the average depth, maximum depth, or minimum depth.

The third imaging mode offers strong spatial penetration of ultrasonic waves, making it suitable for scenarios such as education.

In some embodiments, the processor 40 can automatically set the current imaging mode, such as setting it to the first, second, or third imaging mode.

In some embodiments, the processor 40 obtains the current scanning mode and automatically sets the current imaging mode based on it. Scanning modes include, for example, abdominal scanning mode, liver scanning mode, gallbladder and biliary tract scanning mode, pancreas scanning mode, spleen scanning mode, and kidney scanning mode. These scanning modes can be pre-associated with specific imaging modes. For instance, scanning modes that include far-field imaging regions can be associated with the second imaging mode, while those that only include near-field imaging regions can be associated with the first imaging mode. Accordingly, the processor 40 obtains the current scanning mode, retrieves the associated imaging mode, and sets it as the current imaging mode.

In some embodiments, the processor 40 acquires the current imaging parameters and automatically sets the current imaging mode based on these parameters, with the imaging parameters including at least the imaging depth. In some embodiments, the imaging depth may refer to the average depth or the maximum depth, etc. For example, when the imaging depth is determined to be less than a first value based on the imaging parameters, the processor 40 selects the first imaging mode as the current imaging mode; otherwise, it selects the second imaging mode.

In some embodiments, the processor 40 automatically identifies the scanning region based on the current ultrasound image and switches the current imaging mode accordingly. The processor 40 is capable of recognizing the scanning region from the ultrasound image using traditional image algorithms or relevant machine recognition algorithms. Different scanning regions can be pre-associated with corresponding imaging modes.

The ultrasonic imaging apparatus 100 described herein features an ultrasound probe 10 with array elements operating at two frequencies. In some embodiments, at least a first imaging mode and a second imaging mode are introduced. The first imaging mode offers higher image resolution, while the second imaging mode provides stronger spatial penetration of ultrasound waves, catering to various scenarios and needs. This disclosure addresses requirements such as balancing near-field and far-field imaging with a single ultrasound probe 10, reducing costs and enhancing practicality and ease of use compared to solutions involving multiple probes or dual transducers.

This disclosure describes, with reference to various exemplary embodiments. However, those skilled in the art will recognize that modifications and changes may be made to the exemplary embodiments without departing from the scope of this document. For instance, various operational steps, as well as the components used to execute these steps, may be implemented in different manners, taking into account specific applications or various cost functions related to system operation (e.g., one or more steps may be omitted, modified, or combined with other steps).

Although the principles of this disclosure have been exemplified in various embodiments, numerous modifications to structures, arrangements, proportions, elements, materials, and components, particularly suited to specific environments and operational requirements, may be employed without departing from the principles and scope of this disclosure. Such modifications, as well as any other changes or amendments, are encompassed within the scope of this disclosure.

The foregoing has been described with reference to various exemplary embodiments. However, those skilled in the art will appreciate that various modifications and changes may be made without departing from the scope of this disclosure. Therefore, the disclosure is to be considered illustrative rather than restrictive, and all such modifications will be included within its scope. Similarly, the advantages, solutions to problems, and other benefits of the various embodiments have been described above. However, it should be understood that the benefits, advantages, solutions to problems, and any elements contributing to them, or making them more explicit, are not to be construed as critical, essential, or necessary features of the invention. The term ‘comprising,’ as used herein, and any variants thereof, are intended to be non-exclusive and inclusive, so that a process, method, article, or apparatus that includes a list of elements includes not only these elements but also other elements that are not explicitly listed or that do not belong to the process, method, system, article, or apparatus. Furthermore, the term ‘coupled’ and any other variants thereof, as used herein, refer to physical connection, electrical connection, magnetic connection, optical connection, communication connection, functional connection, and/or any other connection.

Those skilled in the art will recognize that numerous changes can be made to the details of the foregoing embodiments without departing from the fundamental principles of this invention. Therefore, the scope of this invention should be determined solely by the claims.

What is claimed is:

1. An ultrasonic imaging apparatus, comprising:
an ultrasonic probe comprising a transducer array having a plurality of transducer elements with first center frequency and a plurality of transducer elements with second center frequency;
a transmission control circuit configured to control the transducer array to transmit ultrasonic waves;
a reception control circuit configured to control the transducer array to receive echo signals from ultrasonic waves; and
a processor configured to generate an ultrasonic image based on the echo signals;
wherein the ultrasonic imaging apparatus operates in a first imaging mode or a second imaging mode; wherein:
under the first imaging mode, the transmission control circuit controls the transducer elements with first center frequency to operate as transmitting elements to transmit ultrasonic waves to a first region, the reception control circuit controls part or all of transducer elements within the transducer array to operate as receiving elements to receive echo signals from the first region; and the processor generates an ultrasonic image for the first region based on the received echo signals; and
under the second imaging mode, the transmission control circuit controls both the transducer elements with first center frequency and the transducer elements with second center frequency to jointly operate as transmitting elements to transmit ultrasonic waves to a second region, the reception control circuit controls part or all of transducer elements within the transducer array to operate as receiving elements to receive echo signals from the second region, and the processor generates an ultrasonic image for the second region based on the received echo signals.

2. The ultrasonic imaging apparatus of claim 1, wherein, the second region comprises a first sub-region proximal to the transducer array and a second sub-region distal to the transducer array; and
under the second imaging mode, the transmission control circuit controls the transducer elements with first center frequency to operate as the transmitting elements to transmit ultrasonic waves to the first sub-region, and controls the transducer elements with second center frequency to operate as the transmitting elements to transmit ultrasonic waves to the second sub-region.

3. The ultrasonic imaging apparatus of claim 2, wherein, under the second imaging mode, the reception control circuit controls the transducer elements with first center frequency to operate as the receiving elements to receive echo signals from the first sub-region, and controls the transducer elements with second center frequency to operate as the receiving elements to receive echo signals from the second sub-region.

4. The ultrasonic imaging apparatus of claim 1, wherein, an imaging depth of the first region is shallower than that of the second region, and the imaging depth is either an average depth or a maximum depth.

5. The ultrasonic imaging apparatus of claim 1, wherein,
under the first imaging mode, the reception control circuit controls the transducer elements with first center frequency to operate as the receiving elements to receive the echo signals from the first region; and/or
under the second imaging mode, the reception control circuit controls the transducer elements with first center frequency and the transducer elements with second center frequency to jointly operate as the receiving elements to receive the echo signals from the second region.

6. The ultrasonic imaging apparatus of claim 1, wherein,
under the first imaging mode, the transmission control circuit generates excitation signals in a first frequency range to activate the transducer elements with first center frequency; and/or,
under the second imaging mode, the transmission control circuit generates excitation signals in a second frequency range to activate the transducer elements with first center frequency and the transducer elements with second center frequency; or, under the second imaging mode, the transmission control circuit generates excitation signals in a first frequency range to activate the transducer elements with first center frequency, and generates excitation signals in a second frequency range to activate transducer elements with second center frequency.

7. The ultrasonic imaging apparatus of claim 1, wherein the ultrasonic imaging apparatus further operates in a third imaging mode; wherein,
under the third imaging mode, the transmission control circuit controls the transducer elements with second center frequency to operate as the transmitting elements to transmit ultrasonic waves to a third region, the reception control circuit controls part or all of transducer elements within the transducer array to operate as the receiving elements to receive echo signals from the third region, and the processor generates an ultrasonic image for the third region based on the echo signals from the third region.

8. The ultrasonic imaging apparatus of claim 7, wherein, an imaging depth of the first region is shallower than that of the third region, and the imaging depth is an average depth, a maximum depth or a minimum depth.

9. The ultrasonic imaging apparatus of claim 7, wherein, under the third imaging mode, the transmission control circuit generates excitation signals in a second frequency range to activate the transducer elements with second center frequency.

10. The ultrasonic imaging apparatus of claim 1, wherein the processor automatically sets a current imaging mode.

11. The ultrasonic imaging apparatus of claim 10, wherein,
the processor obtains a current scanning mode and automatically sets the current imaging mode based on the current scanning mode; and/or,
the processor obtains current imaging parameters and automatically sets the current imaging mode based on the current imaging parameters, the imaging parameters at least comprising an imaging depth; and/or,
the processor automatically identifies scanned region based on a current ultrasonic image and automatically switches the current imaging mode based on the identified scanned region.

12. The ultrasonic imaging apparatus of claim 1, wherein the transducer array consists of an array of transducer elements arranged in M rows and N columns, where M is an integer greater than or equal to 3, N is an integer greater than or equal to 2; wherein M is an odd number, and for each column of transducer elements: transducer element positioned in the (M+1)/2-th row is individually wired to a channel to receive excitation signals, while transducer elements that are symmetrically positioned around the element positioned in the (M+2)/2-th row of that column are paired, connected together, and then wired to receive excitation signals from the same channel; and for this M×N array, transducer elements in the (M+1)/2-th row are the transducer elements with first center frequency, and transducer elements in the 1^{st} and M-th rows are the transducer elements with second center frequency.

13. The ultrasonic imaging apparatus of claim 12, wherein transducer elements from the 1^{st} to the m-th rows and from the (M+1−m)-th to the M-th rows are the transducer elements with second center frequency, and transducer elements from the (m+1)-th to the (M−m)-th rows are the transducer elements with first center frequency, where m is a positive integer and less than (M+1)/2.

14. The ultrasonic imaging apparatus of claim 1, wherein the transducer array comprises an array of elements arranged in M rows and N columns, where M is an integer greater than or equal to 3, N is an integer greater than or equal to 2; wherein M is an even number, and for each column of transducer elements: transducer elements symmetrically positioned in that column are paired, connected together, and then wired to receive excitation signals from a same channel; and for this M×N array, transducer elements in the M/2-th row and the (M/2+1)-th row are the transducer elements with first center frequency, and transducer elements in the 1st and M-th rows are the transducer elements with second center frequency.

15. The ultrasonic imaging apparatus of claim 14, wherein transducer elements from the 1^{st} to the m-th rows and from the (M+1−m)-th to the M-th rows are the transducer elements with second center frequency, and transducer elements from the (m+1)-th to the (M−m)-th rows are the transducer elements with first center frequency, where m is a positive integer and less than (M+1)/2.

16. The ultrasonic imaging apparatus of claim 1, wherein the transducer elements with first center frequency and the transducer elements with second center frequency are spaced apart and aligned in a row.

17. The ultrasonic imaging apparatus of claim 1, wherein the first center frequency is higher than the second center frequency.

18. The ultrasonic imaging apparatus of claim 17, wherein, the first center frequency is greater than 7 MHz and less than 15 MHz; and/or the second center frequency is greater than 2 MHz and less than 4 MHz.

19. An ultrasonic imaging apparatus, comprising:
an ultrasonic probe comprising a transducer array having an array of elements arranged in M rows and N columns, where for each column of transducers elements: transducer elements symmetrically positioned in that column are paired, connected together, and then wired to a channel to receive excitation signals, and when there is a single central transducer element in that column, the central transducer element is individually wired to a channel to receive excitation signals; and for this M×N array, there are a plurality of transducer elements with first center frequency and a plurality of transducer elements with second center frequency, and transducer elements connected to a same wire are of a same center frequency;
a transmission control circuit configured to control the transducer array to transmit ultrasonic waves;
a reception control circuit configured to control the transducer array to receive echo signals from the ultrasonic waves; and
a processor configured to generate an ultrasonic image based on the echo signals.

20. The ultrasonic imaging apparatus of claim 20, wherein, for this M×N array, some rows of transducer elements are the transducer elements with first center frequency, and other rows of transducer elements are the transducer elements with second center frequency.
